(19) United States
(12) Patent Application Publication
(10) Pub. No.: US 2017/0226153 A1
(43) Pub. Date: Aug. 10, 2017

(54) Method for Separation of Biopolymer Molecules and a Carrier for Application of this Method

(71) Applicant: Univerzita Pardubice, Pardubice-Polabiny (CZ)

(72) Inventors: Rudolf Kupcik, Radimer 425 (CZ); Zuzana Bilkova, Sezemice 38 (CZ); Pavel Rehulka, Dobruska (CZ); Jan Macak, Pardubice (CZ)

(21) Appl. No.: 15/501,020

(22) PCT Filed: Jul. 27, 2015
(86) PCT No.: PCT/CZ2015/000081

§ 371 Date: Feb. 1, 2017

(30) Foreign Application Priority Data
Aug. 1, 2014 (CZ) PV 2014-524

(51) Int. Cl. C07K 1/22; C07H 21/04; B01D 15/42; B01J 20/28; B01D 15/38; C07H 1/06; B01J 20/06

(52) U.S. Cl. CPC C07K 1/22; C07H 1/06; C07H 21/04; B01J 20/06; B01J 20/28009; B01D 15/388; B01D 15/422; B01D 15/3804; C07K 2319/21

(57) ABSTRACT

The invention relates to a method for separation of biopolymer molecules, particularly biopolymer molecules from the group consisting of mono- a multi-phosphorylated peptides, recombinant peptides/proteins with a polyhistidine tag (His-tag) or with another chemically similar biospecific tag, cysteine-containing peptides/proteins and nucleic acids, in which a biopolymer molecule is bound in a binding solution by a specific binding to a carrier, which contains a core with dimensions in nano- and/or submicro- and/or microscale, which is composed of oxide of at least one transition metal and/or silicon oxide, on whose surface is deposited at least one continuous or non-continuous layer and/or nanoparticles of magnetic metal oxide and/or such nanoparticles are deposited in its inner structure, and subsequently undesirable and non-specifically bound components are washed off at least once from the carrier-bound bio-molecules by a washing solution, whereupon biopolymer molecules are eluted from it by changing pH and/or by using an elution solution. The invention also relates to a carrier for application of this method.

### TECHNICAL FIELD

The invention relates to a method for separation of biopolymer molecules.

The invention also relates to a carrier for the application of this method.

### BACKGROUND ART

Nowadays, various methods and various carriers are routinely used for separation of biopolymer molecules, such as mono- and multi-phosphorylated peptides, recombinant peptides/proteins with a polyhistidine tag (His-tag) or another chemically similar biospecific tag, cysteine-containing peptides/proteins and nucleic acids. Nevertheless, these methods and carriers, besides indisputable utility values, also have a number of limiting characteristics—see below.

### Separation of Mono- and Multi-Phosphorylated Peptides

The functions of phosphorylation of proteins and their dynamic changes under physiological conditions have been already described in great detail. However, as has been found over the last 20 years, changes in phosphorylation of proteins relating to certain pathological states in the human body are diagnostically and prognostically very important. Apparatuses for structural analysis of these diagnostically important molecules, including the degree and localization of phosphorylation, are currently in great demand.

Due to the fact that mono- and multi-phosphorylated peptides/proteins are found in cells in very low concentrations, their content dramatically changes with time and their stability during the preanalytical phase is not ideal, it is necessary to use for their separation an effective enrichment technique which considerably increases their relative content in the mixture.

For the purpose of the enrichment of mono- and multi-phosphorylated peptides/proteins from biological materials, a separation technique based on chromatography on immobilized metal ions (IMAC) with Fe³⁺ or Ga³⁺ (Zr⁴⁺, Ti⁴⁺ etc.) was used for a long time—see e.g. the article by Anderson L., Porath J.: “Isolation of phosphoproteins by immobilized metal (Fe⁺³) affinity chromatogramy”, Analytical Biochemistry. 1986, vol. 154, p. 250-254, and the article by Zhou H.: “Zirconium phosphonate-modified porous silicon for highly specific capture of phosphopeptides and MALDI-TOF MS analysis,” Journal of Proteome Research, 2006; vol. 5, p. 2431-2437. It is based on the principle of the interaction between a negatively charged phosphate group and positively charged metal ions which, at the same time, can be bound with the aid of iminodiacetic acid (IDA), nitrilotriacetic acid (NTA) or tris(carboxymethyl)ethylenediamine. However, during the separation of phosphopeptides by means of this technique (IMAC with Fe³⁺ or Ga³⁺), also peptides containing aspartic or glutamic acid or chains containing histidine of a higher density are separated—see e.g. the article by Schling M & Knapp D. R.; “Enrichment of Phosphopeptides Using Biphasic Immobilized Metal Affinity-Reversed Phase Microcolumns”, Journal of Proteome Research, 2008, vol. 7, p. 4164-4172. Furthermore, this non-specific binding increases undesirable contamination of the sample intended for mass analysis.

Reduction of this non-specific binding by methylesterification of carboxyl groups has been described in the article by Haydon C. E. et al. “Identification of Novel Phosphorylation Sites on Xenopus laevis Aurora A and Analysis of Phosphopeptide Enrichment by Immobilized Metal-affinity Chromatography”, Molecular & Cellular Proteomics, 2003, vol. 2, p. 1055-1067.

Another possibility of reduction of this non-specific binding is according to the article Seeley, E. H., et. al.: “Reduction of non-specific binding in Ga^{(III)} immobilized metal affinity chromatography for phosphopeptides by using endoproteinase glu-C as the digestive enzyme”, Journal of Chromatography B, 2005, vol. 817, p. 81-88, cleavage of a sample of peptides/proteins before separation—apart from by trypsin—also by another type of protease, such as enzym Glu-C, which cleaves peptides/proteins behind aspartic and glutamic acids, by which means peptides with acetic amino acid at the C-end are obtained. However, these methods are extremely elaborate and not always sufficiently effective and, what is more, esterification of the residue of aspartic acid worsens interpretability of the spectrum. The drawback of using protease Glu-C is the formation of peptide chains which are too long.

Similar interactions as those between the carrier and Fe³⁺ ions are also used by a carrier based on pure Fe₃O₄— see, e.g., Aera, L., et. al.: “Enrichment of phosphopeptides using bare magnetic particles”, Rapid Communications in Mass Spectrometry, 2008; vol. 22, p. 2561-2564.

Another, although less used, alternative is Al(OH)₃-based affinity chromatography—see, e.g., Wolschin, F.: “Enrichment of phosphorylated proteins and peptides from complex mixtures using metal oxide/hydroxide affinity chromatography (MOAC)”, Proteomics, 2005, vol. 5, p. 4389-4397, in which, however, it is necessary to use different binding conditions from those in the case of metal oxides.

At present, it is especially metal oxides that are used for the specific enrichment of monophosphorylated or diphosphorylated peptides. Most often this relates to porous microparticles of TiO₂, whose size varies in units of micrometers—see, e.g., Jiang Z. T., et. al.: “Synthesis of Porous Titania Microspheres for HPLC Packings by Polymerization-Induced Colloid Aggregation (PICA)”, Analytical Chemistry, 2001, vol. 73, p. 6886-688. Preferably, these microparticles can be superparamagnetic, which brings a number of advantages, such as rapid separation of particles from the liquid phase by means of a strong magnet, zero loss of the carrier or sample, batch off-column configuration or separation occurring in a column in the so-called dynamic fluidized bed. The disadvantage of this technique is the fact that in the case of multi-phosphorylated peptides there are substantial losses caused by a very strong interaction between phosphate groups and microparticles of TiO₂, when the elution of multi-phosphorylated forms of peptides is very low or does not occur at all—see, e.g., Tingholm T. E., et al. “SIMAC (Sequential Elution from IMAC), a Phosphoproteomics Strategy for the Rapid Separation of Monophosphorylated from Multiply Phosphorylated Peptides”, Molecular & Cellular Proteomics 7, 661-671, 2008. Another drawback of this technique is the fact that the microparticles of TiO₂ (or of another transition metal and SiO₂) cannot be used repeatedly, since their binding capacity becomes considerably lower whenever they are used again. Moreover, even before being used for the first time, they have a relatively small specific surface, which is responsible for low initial binding capacity of the carrier.

An exception is represented by magnetically active nanoparticles TiO₂ ranging in size from 100 to 200 nm with trade name MagPrep TiO₂, known, e.g., from WO 2009/115176, which contain a magnetic core composed of Fe₃O₄, which is covered by a coat made of TiO₂.

The article by Chen, C.-T. & Chen, Y.-C.: “Fe₃O₄/TiO₂ Core/Shell Nanoparticles as Affinity Probes for the Analysis of Phosphopeptides Using TiO₂ Surface-Assisted Laser Desorption/Ionization Mass Spectrometry”, Analytical Chemistry 77 (2005), 5912-5919, further deals with the use of laboratory prepared TiO₂ nanoparticles with magnetically active cores composed of Fe₃O₄ for the enrichment of phosphopeptides and subsequent SALDI-MS (mass spectrometry ionization laser attended surface).

The drawback of these nanoparticles is the fact that on their surface they only contain TiO₂, due to which the effect of their magnetic parts has been considerably weakened and, furthermore, specific binding interactions between TiO₂ and the magnetic oxide are not maintained.

Separation of Polyhistidine-Tagged (His-Tag) Recombinant Peptides/Proteins

A number of important bioactive substances that are injected into the human body as a biopharmaceutical, such as insulin, erythropoetin, interferons, subunit vaccines, etc., are currently prepared via a recombinant technique—i.e. using a procedure when into a genome of a producing organism (e.g. bacteria, yeasts, animal cells) is artificially incorporated a vector with a gene encoding the target protein, due to which this protein is expressed along with the native proteins of the cell. At the same time, recombinant protein either accumulates in the cytosol of the cell or is released into the extracellular space or, under certain conditions, it is closed in the so-called inclusion bodies. In any case, the result is, from a chemical point of view, a highly complex mixture of heterogeneous substances. Owing to the fact that even a minimal contamination of recombinant proteins by nonfunctional fragments or by other protein-like substances can cause significant health problems, the requirements for the purity of these proteins for in vivo use in medicine are extremely high. Therefore, for their separation it is necessary to use an effective and highly selective separation method which enables to separate a particular recombinant protein from the other abundant proteins and other chemical substances derived from the producing organism.

Gradually, many different modifications of recombinant proteins have been described and put into practice to facilitate their separation and purification. The most widely used modification is polyhistidine-tagged modification (His-tag), which is composed mostly of 6-8 consecutive histidines at the C- or N-terminus of a polypeptide chain. By means of this tag and its biospecific interaction with a ligand on the carrier, recombinant protein can be readily separated in a relatively pure form from a mixture of many other proteins. At present, in a vast majority of applications, the technique IMAC is used for separation of these proteins, i.e. chromatography on immobilized metal ions of the carrier (see above) with divalent metal ions, of which the most widely used ion is Ni²⁺, less frequently Co²⁺; alternatively, also, for example, Cu²⁺ or Zn²⁺ or other ions can be used. The shortcoming of these carriers is the fact that they are often prepared on the base of cross-linked gels or other polymeric materials which have a low mechanical and chemical resistance. Also common is undesirable ion release from the carrier, for example, in acidic environments. Probably the most important disadvantage of using the technique IMAC for separation of recombinant proteins is the fact that beside polyhistidine, also other amino acids have affinity to the carrier being used (Glu, Asp, Arg, Lys, Tyr, Cys and Met) as well as N-terminal amino group of protein, therefore in order to obtain high purity of the separated protein, it is necessary to appropriately set the equilibrium, washing and elution conditions, which requires considerably experienced operators, and it is not always possible to achieve the desired purity of the isolated protein.

### Separation of Nucleic Acids

Separation of nucleic acids is an essential step in the study of genetic information of many organisms. Molecular diagnostics of infectious diseases, genetic analysis or profiling, as well as gene therapy—these are some examples, when nucleic acids must be—prior to the analysis itself—separated carefully from the cells and isolated also from other contaminating substances present in the cell lysate.

Nowadays, a very common method for separating nucleic acids is using phenol and chloroform—see, e.g., Albariflo C. G., Romanowski V.: “Phenol extraction revisited: a rapid method for the isolation and preservation of human genomic DNA from whole blood”, Molecular and Cellular Probes 8 (1994) 423-427, and the article Manoj C. et al.: “A method for the extraction of high-quality RNA and protein from single small samples of arteries and veins preserved in RNAlater”, Journal of Pharmacological and Toxicological Methods, Volume 47, Issue 2, March-April 2002, 87-92. It is a multi-step process which enables to separate nucleic acids in high purity and with sufficient efficiency, since phenol causes practically all proteins to precipitate so that they can be afterwards easily removed. Also, by adjusting the value of pH to the reaction environment it is possible to perform at the same time specific separations of a certain type of nucleic acids, when, e.g., acidic phenol is used for separation of ribonucleic acids, phenol is used for separation of deoxyribonucleic acids phenol at a pH in the range of 7.5 to 8 and contaminating ribonucleic acids are degraded by RNases, etc. After the separation and after all the purification steps it is often necessary to concentrate nucleic acids for other experiments.

Another method for separation of nucleic acids is using a glass fiber matrix in the presence of high concentrations of chaotropic salts to capture them and subsequently nucleic acids are eluted by a solution with a low concentration of chaotropic salt—see, e.g., Höss M. and Pääbo S.: “DNA extraction from Pleistocene bones by a silica-based purification method”, Nucleic Acids Research 21 (1993) 3913-3914. Most of commercial kits for the extraction of nucleic acids are based on this principle—e.g. DNeasy Blood&Tissue Kit from the company QIAGEN, where the basic material for separation is a silica gel membrane. Similarly, also magnetic carriers are based on this principle, namely, e.g., MagPrep Silica Particles (EMD Milipore). The disadvantage of these kits is their higher price and often also their lower yield.

In a preferable variant, it is also possible to use magnetic particles covered by SiO₂, which can be, moreover, modified, for example, by an amino group—see, e.g., Bal Y. et al. “A rapid method for the detection of foodborne pathogens by extraction of a trace amount of DNA from raw milk based on amino-modified silica-coated magnetic nanoparticles and polymerase chain reaction”, Analytica Chimica Acta 787 (2013) 93-101.

Another method for separating nucleic acids is using ion-exchange liquid chromatography, when the nucleic acid is captured on positively charged particles of the carrier and is subsequently eluted by increasing salt concentration, which results in the gradual release not only of the molecules of nucleic acid, but also of other negatively charged molecules. In this manner, it is possible to separate substances differing in the amount and density of the negative charges on their surface. The disadvantage of this method is its low yield and specificity, the final product being always contaminated with high concentrations of salts.

The article by Amano T., et. al.: “Preparation of DNA-adsorbed TiO₂ particles—Augmentation of performance for environmental purification by increasing DNA adsorption by external pH regulation” Science of the Total Environment 408 (2010) 480-485 discloses a method for separation of nucleic acids on the base of TiO₂, which is based on affinity of the phosphate group of nucleic acids to TiO₂-based materials.

From KR 20070062940 is also known a method for separation of deoxyribonucleic acids for analytical use by means of non-magnetic mesoporous materials made of TiO₂.

The most important considerations when choosing a method for separating deoxyribonucleic acids include the aspect of compatibility of the chosen method with the subsequent PCR (polymerase chain reaction) technique. The polymerization of nucleic acids itself takes place in the range of pH from 8.3 to 8.8 in 10 mM of Tris-HCl with the addition of MgCl₂ and KC. However, also other buffer systems can be used, although it is necessary to maintain functional activity of a particular polymerase under these conditions (e.g. Taq polymerase has an optimum pH of 7.8 to 9.0).

Most of the above-mentioned techniques carried out on a solid phase use the principle of non-specific sorption of nucleic acids, based on physicochemical principles.

### Separation of Cysteine-Containing Peptides/Proteins

In the article by Tambor V., et al.: “CysTRAQ—A combination of iTRAQ and enrichment of cysteinyl peptides for uncovering and quantifying hidden proteomes”, Journal of Proteomics 75 (2012) 857-867 it was experimentally confirmed that up to 15% of peptides in the range of 800 to 3000 Da generated after cleavage of all human proteins by trypsin would contain at least one cysteine. Protein domains, which are rich in cysteines, in fact play a fundamental role in many cellular processes, since they constitute a part of many enzymes, receptors and signalling molecules. Their selective separation is one of the strategies enabling to study primary and secondary structures of proteins.

Peptides/proteins containing thiols, such as glutathione or phytochelatins, play an important role in defending organism against intoxication with heavy metals and provide a balance between oxidation and deoxidation. Glutathione is a tripeptide composed of amino acids of glutamic acid, cysteine and glycine. It is found in the cells of animals, plants and bacteria and protects an organism from oxidative stress. Phytochelatins are post-translationally synthesized peptides, which are found primarily in plants and which effectively bind heavy metals ions, thus protecting the plant from their toxic effects. Cysteines are also often subject to post-translational modifications, such as S-nitrosylation and subsequent oxidation, which results in poor conformation of proteins, which may lead to the formation of aggregates, e.g. in neurodegenerative diseases—see, e.g., the article P. J. Muchowski: “Modulation of neurodegeneration by molecular chaperones”, Nature Reviews Neuroscience 6 (2005), 11-22.

Also venomous spiders, snakes, scorpions or marine snails produce a great variety of cysteine-rich bioactive peptides, which are involved in the stabilization of their structure by a plurality of disulfide bonds. Another major group of cysteine-rich substances includes hydrophobins as primary metabolites of microscopic filamentous fungi, which cause considerable damage to the processes of brewing and malting, especially due to the fact that they cause the beer to produce excess foam—see, e.g., the article Under, M. B., et. al: “Hydrophobins: the protein-amphiphiles of filamentous fungi”, FEMS Microbiology Reviews 29 (2005), 877-896. An example of a clinically significant function of cysteine-rich peptides is association with Parkinson's disease, when GPR37 cysteine-rich domain contributes considerably to cytotoxic effects inside the cell, such as endoplasmic reticulum stress, which results in cell apoptosis—see, e.g., Jorge Gandia et al, “The Parkinson's disease-associated GPR37 receptor-mediated cytotoxicity is controlled by its Intracellular cysteine-rich domain”, Journal of Neurochemistry 125 (2013), 382-372.

So far, several methods for specific separation of cysteine-containing peptides/proteins have been developed, such as covalent chromatography based on specific separation of peptides/proteins with a free sulfanyl group by means of a carrier containing reactive 2-pyridyl disulfide. During this process, 2-pyridyl disulfide reacts with —SH group of peptide, which gives rise to a stable disulfide bond between protein and the carrier and releasing 1,2-dihydro-2-pyridinethione. To elute a covalently-bound molecule, reducing β-mercaptoethanol or dithiothreitol are used—see, e.g., the article Brandt J. et al., Journal of Solid-Phase Biochemistry 2 (1977) 105-109, and the article Liu T. et al.: “Improved proteome coverage by using high efficiency cysteinyl peptide enrichment the human mammary epithelial cell proteome”, Proteomics 5 (2005), 1263-1273.

Among commercial products which are used for separation of cysteine-rich proteins or separation of peptides containing cysteine, the most commonly used one is Thiopropyl Sepharose 6B or 4B (GE Healthcare), a carrier containing 2-pyridyl disulfide having a size range between 45 and 165 μm.

Other methods, too, are based on a similar principle. These methods serve to label the specific —SH group of cysteine, when the complexity of the initial material to be analyzed is reduced, whereby the labelled molecule also allows the substance to be quantified.

The article by Spahr C. S., et. al.: “Simplification of complex peptide mixtures for proteomic analysis: reversible biotinylation of cysteinyl peptides”, Electrophoresis 21 (2000), 1635-1650 discloses a technique of reversible biotinylation of cystein peptides, the article Giron P, et at: “Cysteine-reactive covalent capture tags for enrichment of cysteine-containing peptides”, Rapid Communications in Mass Spectrometry 23 (2009), 3377-3386 describes the use of cysteine-reactive tags for covalent capture, the article Gygi S. P., et al.: “Quantitative analysis of complex protein mixtures using isotope-coded affinity tags”, Nature Biotechnology 17 (1999), 994-999, describes an approach called ICAT, and the article Tambor V., et al.: CysTRAQ—a combination of iTRAQ and enrichment of cysteinyl peptides for uncovering and quantifying hidden proteomes”, Journal of Proteomics 75 (2012) 857-867, deals with cysteine-reactive tags for tandem mass spectrometry and CysTRAQ.

The goal of the invention is to eliminate the disadvantages of the background art and propose a method for efficient separation and purification of biopolymer molecules which would be suitable for the majority of significant biopolymer molecules and some selected biotechnological and biomedical applications.

In addition, the goal of the invention is to propose a carrier to be used when applying this method.

### Principle of the Invention

The goal of the invention is achieved by a method for separation of biopolymer molecules, particularly biopolymer molecules from the group consisting of mono- and multi-phosphorylated peptides, recombinant peptides/proteins with a polyhistidine tag (His-tag) or with another chemically similar biospecific tag, cysteine-containing peptides/proteins and nucleic acids, whose principle consists in that a biopolymer molecule is bound in a binding solution by a specific binding to a carrier, which contains a core with dimensions in nano- and/or submicro- and/or microscale, composed of oxide of at least one transition metal and/or silicon oxide, on whose surface is deposited at least one continuous or non-continuous layer and/or nanoparticles of magnetic metal oxide and/or such nanoparticles are deposited in its inner structure and subsequently undesirable and non-specifically bound components are washed off at least once from the carrier-bound biomolecules by a washing solution, whereupon the required biopolymer molecules are eluted from it by changing pH and/or by using an elution solution. In this manner, high purity of the separated biopolymer molecule is obtained.

It is advantageous if the binding solution for binding mono- and multi-phosphorylated peptides to the carrier contains 35 to 90% of organic solvent and 0.1 to 5% of carboxylic acid. Preferably, the washing solution contains 5 to 90% of organic solvent and 0.1 to 5% of carboxylic acid. So as to reduce the non-specific binding, the binding and/or washing solution can also contain 0.5 to 3 M of different carboxylic acid. Mono- and/or multi-phosphorated peptide is then eluted from the carrier with an elution solution at a pH higher than 9, or by competitive elution with a solution containing at least 20 mM of phosphate/phosphates.

Preferably, the binding solution for binding recombinant peptide/protein with a polyhistidine tag (His-tag) or another chemically similar biospecific tag to the carrier contains any buffer and 0-300 mM of imidazole, its pH ranging between 3 and 8.7. The washing solution, too, contains any buffer and 0-300 mM of imidazole, whereby its composition is preferably the same as that of the binding solution. In order to reduce the non-specific binding, the binding and/or washing solution can contain up to 0.5 M of salt/salts. Recombinant peptide/protein with a polyhistidine tag (His-tag) or another chemically similar biospecific tag is afterwards eluted from the carrier by changing pH to 9.5-12. Preferably, recombinant protein is eluted from the carrier with an elution solution containing at least 20 mM of phosphate/phosphates or additional imidazole up to the total amount of imidazole 10 mM-1 M, recombinant peptide is then eluted by increasing pH in the range from 9.5 to 12 or with an elution solution containing at least 20 mM of phosphate/phosphates.

The binding solution for binding cysteine-containing peptides/proteins to the carrier contains any buffer and 0-2% of sodium dodecyl sulfate, its pH being 6-8. Also the washing solution contains any buffer and 0-2% of sodium dodecyl sulfate, with pH 6-8, whereby its composition Is preferably the same as that of the binding solution. In order to reduce the non-specific binding, the binding and/or the washing solution can contain 10-50 mM of dithiothreitol, mercaptoethanol or mercaptobenzoic acid. Cysteine-containing peptide/protein is eluted from the carrier by changing pH to a value of 10 to 12, e.g. by additional phosphate/phosphates.

The binding solution for binding deoxyribonucleic acid to the carrier is composed of a buffer with pH 4.5-6.5. Also the washing solution is in this case composed of a buffer with pH 4.5-6.5, whereby in a preferable variant of embodiment its composition is the same as that of the binding solution.

The binding solution for binding ribonucleic acid to the carrier is composed of a buffer with a pH from 3.8 to 4.5. Also the washing solution is in this case composed of a buffer with a pH from 3.8 to 4.5, whereby in a preferable variant of embodiment its composition is the same as that of the binding solution.

Deoxyribonucleic acid or ribonucleic acid is afterwards eluted from the carrier by increasing pH to a value from 8 to 11, or with an elution solution containing at least 20 mM of phosphate/phosphates.

In all the variants of separation of biopolymer molecules according to the invention, it is advantageous to use a carrier which contains a core consisting of TiO₂ nanotubes, on whose surface are deposited Fe₃O₄ nanoparticles.

After the completion of the separation it is advisable to regenerate the carrier that has been used or, optionally, to carry out decontamination and sterilization, namely, e.g., by means of photocatalysis initiated by UV radiation having a wavelength from 200 to 400 nm and an intensity of at least 1 mW/cm² of the area of the carrier.

Beside a method for separation, the goal of the invention is also achieved by a carrier for separation of biopolymer molecules, particularly biopolymer molecules from the group consisting of mono- and multi-phosphorylated peptides, recombinant peptides/proteins with a polyhistidine tag (His-tag) or with another chemically similar biospecific tag, cysteine-containing peptides/proteins and nucleic acids, whose principle consists in that it contains a core having dimensions in nano- and/or submicro- and/or microscale, formed by oxide of at least one transition metal and/or silicon oxide, whereby at least on a part of the surface of the core is deposited a layer and/or are deposited nanoparticles of magnetic metal oxide and/or these nanoparticles are deposited in its inner structure. At the same time, it is advantageous if at least a part of some particles projects to the surface of the core or protrudes from it.

Preferably, the core of such a carrier is composed of silicon oxide and/or oxide of transition metal from the group consisting of TiO₂, ZrO₂, Al₂O₃, Ta₂O₅, WO₃, SnO₂, HfO₂, Nb₂O₅, MoO₂, MoO₃, ZnO, V₂O₅, Fe₂O₃, Fe₃O₄, or of a mixture of at least two of them, whereby magnetic metal oxide is an oxide with a metal from the group consisting of FeₓO_{y}, NiO, CoO, Co₃O₄, ferrite based on M₂O₃, where M is a metal from the group consisting of Fe, Ni, Co, or a mixture of at least two of them.

In the most advantageous alternative, the carrier according to the invention contains a core composed of TiO₂ nanotubes, on whose surface are deposited Fe₃O₄ nanoparticles.

### DESCRIPTION OF DRAWINGS

FIG. 1 of the enclosed drawings shows a 100,000 times magnified photograph of a carrier developed for separation of biopolymer molecules according to the invention in one variant of embodiment,

FIG. 2 shows four flow charts of the method for isolation of four different biopolymer molecules according to the invention,

FIG. 3 shows three MS spectra comparing clean standard—phosvitin cleaved by trypsin, the result of the enrichment of phosvitin with the aid of a commercial carrier known from the background art and the result of the enrichment of phosvitin with the aid of the carrier according to the invention in FIG. 1,

FIG. 4 shows two MS spectra comparing standard—commercial ubiquitin with a His-tag cleaved by trypsin, the result of separation of ubiquitin using the carrier according to the invention in the alternative according to FIG. 1 and finally,

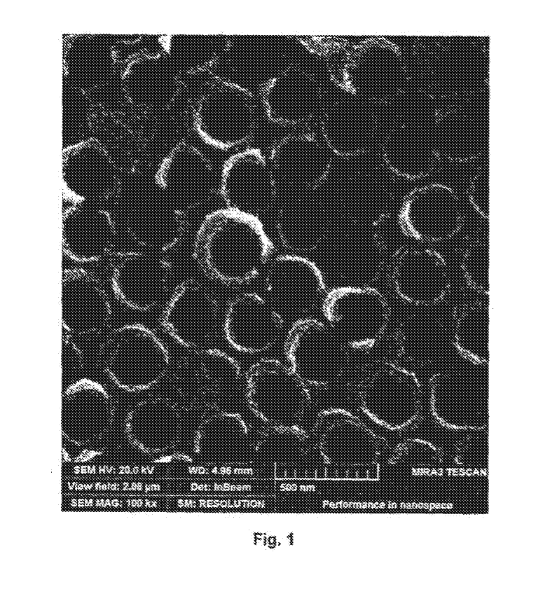

in FIG. 5 there are two MS spectra comparing standard—bovine serum albumin cleaved by trypsin, and the result of separation of peptides from bovine serum albumin by means of the carrier according to the invention in the alternative according to FIG. 1.

### SPECIFIC DESCRIPTION

The method for separation of biopolymer molecules (particularly mono- and multi-phosphorylated peptides, recombinant peptides/proteins with a polyhistidine tag (His-tag) or another chemically similar biospecific tag, cysteine-containing peptides/proteins and nucleic acids) according to the invention is based on using a composite carrier composed of a core consisting of a material based on oxide of transition metal/metals (e.g. Ti, Al, Zr, Ta, Hf, W, Nb, Sn, V, Fe) or silicon oxide, on whose surface and/or in whose inner structure is deposited magnetic metal oxide (e.g. Fe, Co and Ni).

The core of this carrier is composed of substantially any formation, e.g. by a spherical or substantially spherical particle, a tube (a hollow elongated object, whose length is considerably greater than its diameter), a fiber (a solid elongated object, whose length is considerably greater than its diameter), or substantially any other object/particle (regular or Irregular, e.g., a cube, a rectangular parallelepiped, a prism, a pyramid, etc.), having dimensions in nano-, sub micro- or microscale. In particular, its material is transition metal oxide (preferably, e.g., TiO₂, ZrO₂, Al₂O₃, Ta₂O₅, WO₃ SnO₂, HfO₂, Nb₂O₅, MoO₂, MoO₃, ZnO, V₂O₅, Fe₂O₃, Fe₃O₄, or a mixture of at least two of them in any ratios or proportions, or a mixture of both stoichiometric and nonstoichiometric forms of at least one of them in any ratios), silicon oxide (SiO₂— in any ratios of stoichiometric and nonstoichiometric forms), or a mixture of at least one transition metal oxide and SiO₂ in any ratios. Preferably, especially TiO₂ can be used as a material of the core of the carrier as the most biocompatible of all the oxides which have been mentioned above.

On the surface of this core and/or in its inner structure is deposited magnetic metal oxide, preferably especially any stoichiometric or nonstoichiometric variants of FeₓO_{y}, NiO, CoO, Co₃O₄, ferrite based on M₂O₃, where M is a metal from the group consisting of Fe. Ni, Co Fe, Ni, Co. or a mixture of at least two of them. Also, it is advantageous if we use magnetite and/or maghemite, which are not toxic for the human body and which exhibit—supposed they are in the form of particles smaller than 12 nm—superparamagnetic properties. Furthermore, this material can be deposited on the surface of the core in the form of continuous or noncontinuous layers and/or randomly dispersed nanoparticles, and/or is deposited in the form of nanoparticles in its inner structure, ideally in such a manner that at least part of some nanoparticles protrude onto the surface. In the first alternative, nanoparticles of magnetic metal oxide on the surface of the core are preferably tagged by covalent or other physico-chemical interactions which ensure maximum protection from external, especially physical influences (washing off, releasing, etc.), thus ensuring long-term dynamics of a particular carrier in the magnetic field.

The main advantage of the carrier according to the invention is the fact that it enables the specific surface interaction of the transition metal oxide with biopolymer molecules which are to be separated, while maintaining at the same time the contribution of its magnetic material which allows to effectively remove the carrier from the sample due to the action of the magnetic field; moreover, this carrier has a huge specific surface. Another benefit of the carrier according to the invention is its extensive chemical, physical and mechanical compatibility—in different variants there are in principle very similar materials which can be replaced with one another and/or combined.

The most advantageous variant of the carrier is the variant in which its core is composed of a TiO₂ nanotube on whose surface are deposited Fe₃O₄ nanoparticles (FIG. 1), since these materials do not exhibit toxicity for the human organism, are biocompatible and, at the same time, they can be used repeatedly since they sustain high binding ability and selectivity.

In addition, the carrier according to the invention can be effectively decontaminated by using photocatalysis and sterilized by the application of UV light Irradiation.

During the production of the carrier according to the invention, magnetic metal oxide is preferably applied to pre-prepared cores, e.g. by a direct chemical method, using a method of sputtering, a method of atomic layer deposition (ALD—Atomic Layer Deposition).

The carrier thus obtained exhibits both sufficient mechanical and chemical resistance, and so it can be used for efficient separation of biopolymer molecules in which the biopolymer molecules to be separated are specifically bound to the carrier, whereupon undesired and non-specifically bound components from a particular sample are washed off, and finally the biopolymer molecules to be separated are eluted by changing pH or with a suitable solution. Biopolymer molecules separated by this process achieve thanks to this high purity.

Magnetic properties of the composite carrier according to the invention allow a variety of uses—whether it is batch or column arrangement in the form of a magnetically stabilized fluidized bed (Magnetically Stabilized Fluidized Bed—MSFB). Spherical carrier variants can be in a classical column arrangement, whereas fibrous arrangements can be used, for example, for the production of filters which will specifically isolate the target substances. In all these cases, a strong magnetic field is used for the purpose of stabilization of the separation bed and/or efficient extraction of carriers with bound biopolymer molecules from the reaction mixture.

Preferably, the magnetic material deposited on the surface of the core of the carrier and/or in its inner structure has superparamagnetic properties, due to which the carrier after removal of the magnetic field does not exhibit residual magnetism (which ferromagnetic materials would exhibit), and so it is possible to work with it alternately in the mode of homogeneously dispersed suspension or in separate phases.

In another alternative of separation of biopolymer molecules, the carrier that has been described above is incorporated into separation channels of microfluidic systems (the so-called μTAS), by which means the positive effect of miniaturization of reaction volume during proof or separation of a particular biopolymer molecule is multiplied, although the specific surface area is still high due to the nature of the filling of the separation microchannel of the system.

Another alternative of performing the method for separation of biopolymer molecules is capturing a compact block of carriers, whose core is composed of flowthrough nanotubes (their lower closed portion formed during the production being removed), in a holder of a membrane, whereby the mixed solution of the substances flows through the cavities of the nanotubes in a similar manner as in the case of membrane filtration processes.

### Separation of Mono- and Multi-Phosphorylated Peptides

In the initial medium—e.g. in a mixture of peptides/proteins, cell lysate, cell growth medium, etc., the proteins contained are first cleaved into shorter peptide chains. Subsequently, the mono- and multi-phosphorylated peptides thus obtained are bound by a specific interaction between their phosphate group and metal oxide to the carrier according to the invention. The actual process of the binding of the peptides to the carrier takes place in a binding solution which contains 35-90% of organic phase (preferably acetonitrile (ACN), methanol, ethanol or another organic solvent) and 0.1-5% of carboxylic acid (preferably trifluoroacetic acid (TFA), formic acid, etc.), whereby low pH of this solution is used to suppress the non-specific binding. In addition, to reduce the non-specific binding, it is possible to use a different carboxylic acid, e.g. lactic acid (preferably with a concentration of 0.5 to 2 M), glycolic add, salicylic acid, phthalic acid, 2,5-dihydroxybenzoic acid (preferably with a concentration of 0.5 to 3 M), etc.

After the binding, undesirable and non-specifically bound components from the carrier are washed off at least once with a washing solution which contains from 5 to 90% of organic phase (preferably ACN, methanol, ethanol or another organic solvent) and 0.1-5% of carboxylic acid (preferably TFA, formic acid, etc.), or with a binding solution.

After that, specifically bound mono- and/or multi-phosphorylated peptides are released from it with an elution solution with a pH higher than 9 (e.g. 1% aqueous ammonia solution). In another variant, elution can be carried out competitively, using a solution containing phosphate/phosphates (at least 20 mM).

Binding, washing and elution steps are assisted by a magnetic field, when the carrier is separated from the liquid.

The separated mono- and multi-phosphorylated peptides can be further used, e.g. for subsequent (MS) analysis.

After the separation, the carrier of the Invention in case of need is regenerated (see below).

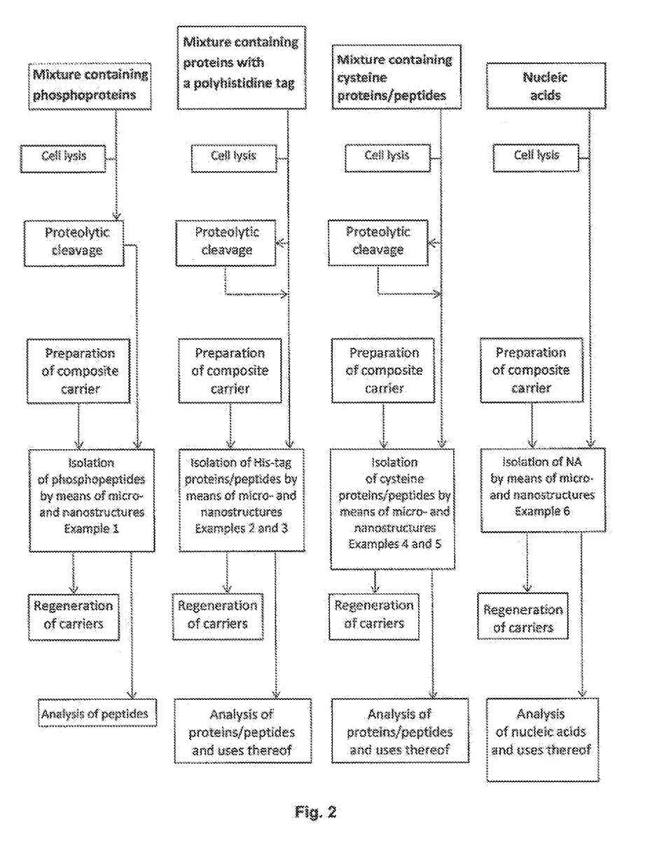

Separation of mono- and multi-phosphorylated peptides is indicated in FIG. 2 in the first flow chart from the left-hand side.

### Example 1

1 mg of the carrier according to the invention, which was previously stored in a solution containing 80% of acetonitrile (ACN) and 0.1% of trifluoroacetic acid (TFA), was washed under the influence of a magnetic field with 500 μl of a washing solution containing 80% of ACN and 0.1% of TFA, and after that with 500 μl of the binding and washing solution containing 80% of ACN, 5% of TFA and 1 M of lactic acid (LA). The excess solution was removed and to the carrier was added 200 μl of a solution containing 60 pmol of proteolytically cleaved phosvitin—model hyperphosphorylated protein, dissolved in a binding and washing solution having the composition as described above. The carrier with applied cleaved phosvitin was afterwards incubated under gentle rotation at room temperature for 60 min.

After incubation, the binding and washing solution containing unbound components was removed from the reaction mixture and the carrier was washed with 2×500 μl of a washing solution containing 80% of ACN, 5% of TFA and 1 M of LA and with 2×500 μl of a washing solution containing 80% of ACN and 0.1% of TFA. 50 μl of an elution solution (1% aqueous ammonia solution) was then added to the carrier thus washed, whereby elution proceeded under rotation for 15 minutes.

Then, after the elution solution above the carrier was removed, it was acidified with TFA and analyzed by means of mass spectrometry (MS).

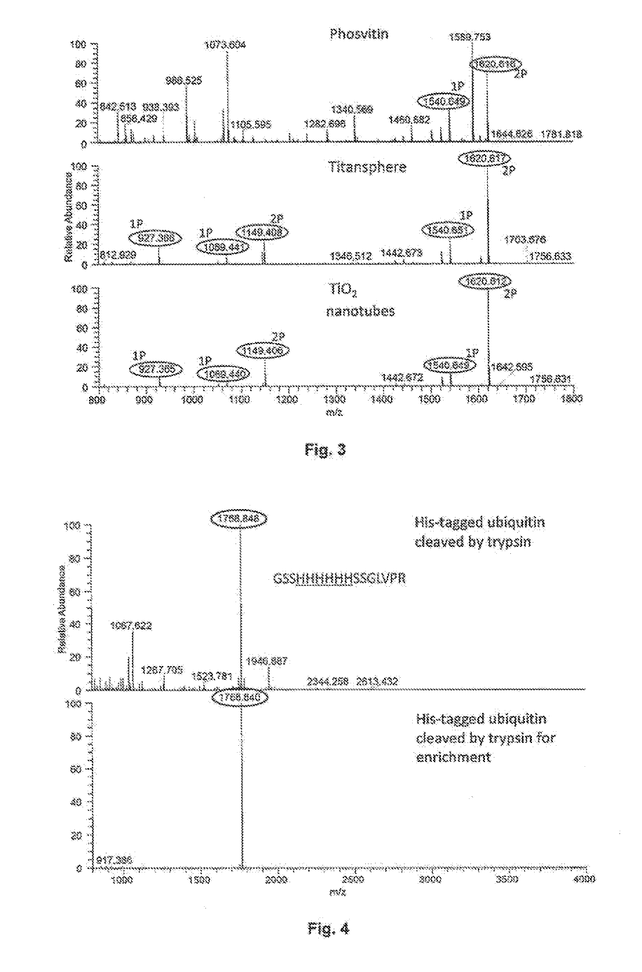

FIG. 3 shows three MS spectra—at the top there is a MS spectrum of a clean standard—phosvitin, in the middle there is a MS spectrum of phosvitin separated by using the most commonly used type of a carrier—particularly Titansphere™ TiO Bulk Material, 10 μm produced by GL Sciences Inc., and at the bottom there is a MS spectrum of phosvitin separated by using the carrier according to the invention in the variant according to FIG. 1, when its core is composed of TiO₂ nanotubes with Fe₃O₄ nanoparticles deposited on their surface. The comparison of these spectra clearly demonstrates that the ratio between the multi-phosphorylated peptides—peaks 1620 and 1149, and the mono-phosphorylated peptides—peaks 1540 and 1069, is when using the carrier according to the invention shifted more towards the multi-phosphorylated peptides. Also, peaks 1540 and 1620, as well as 1149 and 1069, only differ in the amount of phosphogroups and have the same amino acid sequence, which means that the carrier according to the invention separates the multi-phosphorylated peptides better than the carrier Titansphere™ TiO. Moreover, a purer sample is obtained under the same conditions, and when using the carrier according to the invention, MS spectrum has one order higher Intensity, which indicates high capture efficiency, as well as high selectivity.

Owing to the fact that separation according to the invention is based on the specific interaction between a phosphate group mono- or multi-phosphorylated peptide and the metal oxide of the carrier according to the invention, it is apparent that when using the same or similar technique of separation, the other mono- a multi-phosphorylated peptides will behave in the same or similar manner as above-mentioned phosvitin.

### Separation of Peptides/Proteins Containing His-Tag

In the initial medium—e.g. a mixture of peptides/proteins, cell lysate, cell growth medium, etc., for separation at the peptide level at first the contained proteins are proteolytically cleaved into shorter polypeptide chains. For separation at the protein level, the proteins are separated in an intact form. In both cases, the carrier according to the invention is used, whereby separation of peptides/proteins is based on the specific Interaction between positively charged groups around atom N in the structure of histidine with oxide of metal/metals of the carrier.

Preferably, binding of peptides to the carrier occurs in a binding solution containing 10 mM of imidazole in 100 mM of glycin-HCl buffer with pH 3.2, whereby the same solution is then preferably used also for washing off undesirable and non-specifically bound components.

In the case of proteins, for binding to the carrier and for washing off undesirable and non-specifically bound components from the carrier, a solution consisting of 50 mM of Tris-HCl buffer with a pH of 7.5 is preferably used.

In general, it is possible to use a binding and/or washing solution which contains a buffer of any composition in the pH range of 3 to 8.7, with the addition of 0-300 mM of imidazole. Generally, it is possible to use any buffer or any substance which has the given range of pH and at the same time is not an elution agent. For the purpose of suppressing non-specific binding, the solutions can be further supplemented with salts, for example NaCl, up to a concentration of 0.5 M.

Peptides are eluted from the carrier by changing pH in the range between 9.5 and 12 or with elution solution/solutions containing a minimum of 20 mM of phosphate/phosphates (for example, disodium phosphate or a phosphate buffer). Proteins are eluted from the carrier by additional imidazole or increasing its concentration compared to the binding and washing solution (to 10 mM-1 M) or also with elution solution/solutions containing a minimum of 20 mM of phosphate/phosphates (for example, disodium phosphate or a phosphate buffer)

If for some reason it is desirable only to remove peptides from the solution, it is possible to leave them bound to the carrier and eliminate them afterwards during its regeneration. This procedure is applicable, e.g., for removing the cleaved off polyhistidine tags.

The binding, washing and elution steps are assisted by the magnetic field by which the carrier is being separated from the liquid.

After the separation the carrier according to the invention in case of need is regenerated (see below).

Separation of peptides/proteins containing His-tag is indicated in FIG. 2 by the second flow chart from the left-hand side.

### Example 2—Separation of Peptides Containing His-Tag

1 mg of the carrier according to the invention, which was previously stored in a solution containing 80% of ACN and 0.1% of TFA, was washed under the influence of the magnetic field with 2×500 μl of a washing solution consisting of 0.1 M of glycin-HCl buffer with pH 3.2. To the washed carrier was then added 60 pmoles of proteolytically cleaved ubiquitin with a His-tag dissolved in 200 μl of a binding solution composed of 0.1 M of glycine-HCl buffer with pH 3.2. The carrier with applied ubiquitin was then incubated under gentle rotation at room temperature for 60 min.

After incubation, the binding solution with unbound components was removed from the test tube and the carrier was washed with 2×300 μl of a washing solution consisting of 0.1 M of glycin-HCl buffer with pH 3.2, with 2×300 μl of a washing solution containing 0.1 M of glycin-HCl buffer with pH 3.2 and 150 mM of imidazole, and after that with 300 μl of a washing solution consisting of 0.1 M of glycin-HCl buffer with pH 3.2.

100 μl of an elution solution (1% aqueous ammonia solution) was added to the carrier thus washed, whereby elution proceeded under rotation for 15 minutes. After the elution was completed, the elution solution above the carrier was removed, the solution was acidified with TFA and analyzed by means of MALDI-MS.

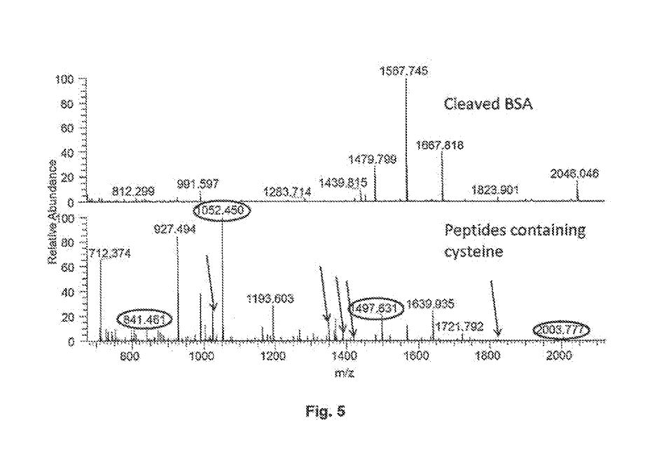

FIG. 4 shows 2 MS spectra—at the top there is a MS spectrum of commercial His-tagged ubiquitin cleaved by trypsin, the lower one is a MS spectrum of ubiquitin separated with the aid of the carrier according to the invention in the alternative according to FIG. 1, when its core is formed by TiO₂ nanotubes with Fe₃O₄ nanoparticles deposited on their surface. It follows from the comparison of the two spectra that during separation almost pure His-tagged ubiquitin was obtained (peak 1768.840) with amino acid sequence GSSHHHHHHSSGLVPR. This confirms high affinity of the carrier according to the invention to recombinant peptides/proteins with a polyhistidine tag.

### Example 3—Separation of His-Tagged Proteins

5 mg of the carrier according to the invention, which was previously stored in a solution containing 80% of ACN and 0.1% of TFA, was washed under the influence of the magnetic field with 2×500 μl of a washing solution consisting of 50 mM of Tris-HCl buffer with pH 7.5. To the washed carrier was then added 200 μl of a binding solution composed of 50 mM of Tris-HCl buffer with pH 7.5, which contained recombinant His-tagged protein. The carrier with the applied proteins was afterwards incubated under gentle rotation at room temperature for 60 min.

After incubation, the binding solution with unbound components was removed from the test tube and the carrier was washed with 5×500 μl of a washing solution consisting of 50 mM of Tris-HCl buffer with pH 7.5.

After that, 2×100 μl of an elution solution containing 50 mM of Tris-HCl buffer with pH 7.5 and 150 mM of imidazole were added to the carrier thus washed, whereby elution proceeded under rotation for a period of 2×15 minutes. After the completion of the elution process, the elution solution above the carrier was removed and both the elution and washing solution were analyzed by means of polyacrylamide gel electrophoresis in sodium dodecyl sulfate environment, whereby it was found that the electrophoretogram of the elution fraction, unlike the original sample, contains pure recombinant protein with minimal impurities.

With regard to the fact that separation according to the invention is based on a specific interaction between the positively charged group around atom N in the structure of histidine with the metal oxide of the carrier according to the invention, it is apparent that the other peptides/proteins with a His-tag or another chemically similar biospecific tag, will behave in the same or similar manner when using the same or similar technique of separation.

### Separation of Cysteine-Containing Peptides/Proteins

In the initial medium—e.g. a mixture of peptides/proteins, cell lysate, cell growth medium, etc., for the separation at the peptide level, at first the proteins contained are proteolytically cleaved into shorter polypeptide chains. For separation at the protein level, the proteins are separated in an intact form. Peptides or proteins with cysteines are then separated using the carrier according to the invention on the basis of the specific interaction between —SH group of cysteine and the metal oxide of the carrier. The free —SH group, if it is no longer present, is obtained by the preceding reduction of —S—S— bonds by means of dithiotreitole (at least 10 mM).

Similarly, cysteine-containing peptides/proteins can be isolated by means of the specific interaction between —SO₃H group and the metal oxide of the carrier. The free —SO₃H group is obtained by a preceding oxidation of —SH group or directly from —S—S— bonds by means of performic acid (at least 2%).

Binding of peptides/proteins to the carrier then takes place in a binding solution containing 100 mM of Tris-HCl buffer with pH 7.5 and 1% of sodium dodecyl sulfate (SDS). In general, it is possible to use a binding solution which contains a buffer of any composition with a pH in the range between 6 and 8 with additional 0-2% of SDS. Generally, it is possible to use any buffer or any substance which has the given range of pH and at the same time is not an elution agent. The same solution is then used also as a washing solution for washing off undesirable and non-specifically bound components from the carrier. For the purpose of suppressing non-specific binding, the binding and washing solution can be further enriched with 10-50 mM of dithiothreitol, mercaptoethanol or mercaptobenzoic acid.

Afterwards, elution is carried out by increasing pH to a value from 10 to 12 or with an elution solution containing at least 20 mM of phosphate/phosphates.

The binding, washing and elution steps are assisted by the magnetic field by which the carrier is being separated from the liquid.

After the separation the carrier according to the invention in case of need is regenerated (see below).

Separation of peptides/proteins containing cysteine is indicated in FIG. 2 by the second flow chart from the right-hand side.

### Example 4

Separation of peptides containing cysteine/cysteines with a free —SH group from a model sample—bovine serum albumin cleaved by trypsin—was carried out with the aid of the carrier according to the invention. 0.5 mg of this carrier, which was previously stored in a solution containing 80% of ACN and 0.1% of TFA, was washed under the influence of the magnetic field with 2×500 μl of a washing solution containing 100 mM of Tris-HCl buffer with pH 7.5 and 1% of sodium dodecyl sulfate (SDS). To the washed carrier was then added a binding solution with 60 pmol of a model sample in 200 μl 100 mM of Tris-HCl buffer with pH 7.5, with additional 1% of SDS. The carrier with the applied sample was afterwards incubated under gentle rotation at room temperature for 60 min.

After incubation, the binding solution with unbound components was removed from the test tube and the carrier was washed with 6×300 μl of a washing solution containing 100 mM of Tris-HCl buffer with pH 7.5 and 1% of SDS.

After that, 100 μl of an elution solution (1% aqueous ammonia solution) was added to the washed carrier, whereby elution proceeded under rotation for a period of 15 min. After the completion of the elution, the elution solution above the carrier was removed and both the elution and washing solution were analyzed by means of mass spectrometry (MS).

FIG. 5 shows 2 MS spectra—the upper one is a MS spectrum of the standard, the lower one is a MS spectrum of peptides separated with the use of the carrier according to the invention in the variant according to FIG. 1, when its core is formed by TiO₂ nanotubes with Fe₃O₄ nanoparticles deposited on their surface. Moreover, cysteine-containing peptides are marked with arrows, whereby, e.g., peak with the highest intensity in the lower MS spectrum (1052.450) with amino acid sequence CCTKPESER is in the upper spectrum completely invisible. That demonstrates the selectivity of the carrier according to the invention preferentially for cysteine-containing peptides.

### Example 5

1 mg of a carrier according to the invention, which was previously stored in a solution containing 80% of ACN and 0.1% of TFA was under the influence of a magnetic field washed with 2×500 μl of a washing solution containing 100 mM of Tris-HCl buffer with pH 7.5 with additional 1% of SDS. To the washed carrier was added a mixture of proteins (60 pmol of each) containing bovine serum albumin, in which —S—S— bonds (disulfide bridges), were reduced by means of 20 mM of dithiothreitol.

This mixture was added to the carrier in 200 μl of a binding solution containing 100 mM of Tris-HCl buffer with pH 7.5 with additional 1% of SDS. The carrier with the applied proteins was afterwards incubated under gentle rotation at room temperature for 60 min.

After incubation, the binding solution with unbound components was removed from the test tube and the carrier was washed with 6×300 μl of a washing solution containing 100 mM of Tris-HCl buffer with pH 7.5 with additional 1% of SDS.

100 μl of an elution solution (1% aqueous ammonia solution) was added to the carrier thus washed, whereby elution proceeded with rotation for a period of 15 min.

After the completion of the elution, the elution solution above the carrier was removed and this solution was acidified (to pH 8). This was followed by cleavage of the separated proteins by trypsin into peptide fragments and the analysis of the elution and washing solution by means of mass spectrometry (MS), whereby almost solely the peptides derived from bovine serum albumin were found in the elution solution.

Given that separation of proteins/peptides with cysteine/cysteines according to the invention is based on the specific interaction between —SH group or —SO₃H group of cysteine and the oxide of metal/metals of the carrier according to the invention, it is evident that the other cysteine-containing peptides/proteins will behave in the same or similar manner if the same or similar technique of separation is used.

### Separation of Nucleic Acids

Chains of deoxyribonucleic acids (DNA) are separated using a carrier according to the invention on the basis of the interaction between phosphate groups, which are part of nucleic acids, and the oxide of metal/metals of the carrier. The binding of DNA to the carrier occurs in a buffer based on the 2-(N-morpholino)ethanesulfonic acid (MES) in the pH range of 4.5 to 6.5. Afterwards, acetate buffer with a pH range of 3.8 to 4.5 is used for separation of ribonucleic acids (RNA). Generally, in both cases it is possible to use any buffer or any substance which, reaching the particular range of pH, is not at the same time an elution agent. Preferably, washing solutions for removing undesirable and non-specifically bound components are in both cases identical with the binding solutions in which the binding to the carrier takes place.

Elution is in both cases performed by increasing pH to a value of 8-11, or by an elution solution containing at least 20 mM of phosphate/phosphates, e.g. disodium phosphate or phosphate buffer.

The analysis of the elution and washing solutions is then carried out e.g. by means of electrophoresis (agarose or polyacrylamide) or by means of polymerase chain reaction (PCR).

Separation of nucleic acids is indicated in FIG. 2 by the first flow chart on the right-hand side.

### Example 6

1 mg of the carrier according to the invention, which was previously stored in a solution containing 80% of ACN and 0.1% of TFA, was under the influence of a magnetic field washed with 2×500 μl of a washing solution containing 100 mM of MES buffer with pH 5.5. To the washed carrier was then added a binding solution containing 10 μg of model deoxyribonucleic acid-oligonucleotide in 100 μl 100 mM of MES buffer with pH 5.0. The carrier with the applied binding solution was afterwards incubated under gentle rotation at room temperature for 60 min.

After incubation, the binding solution with unbound components was removed from the test tube and the carrier was washed with 5×200 μl of a washing solution containing 100 mM of MES buffer with pH 5.5.

100 μl of an elution solution Na₂HPO₃ was then added to the carrier thus washed, elution proceeded under rotation for a period of 15 min. After its completion, both the elution and the washing solution were removed and analyzed by polyacrylamide gel electrophoresis using fluorescent detection, in which it was found that not only can oligonucleotides be bound to the carrier according to the invention, but they can also be easily released from it.

### Regeneration of the Carrier

So as to regenerate the carrier according to the invention with the purpose of reusing it, it is necessary to remove all residues of organic substances from the carrier. In the case of the carrier which contains a TiO₂ core it is possible to use for this purpose preferably irradiation of the carrier by UV radiation in an aqueous medium. TiO₂ in that case generates on its surface highly reactive hydroxyl radicals, which have a considerable ability to cleave organic molecules into elemental carbon dioxide and water, by virtue of which the carrier according to the invention is completely free from any kind of undesirable organic residues. At the same time, however, there is no degradation of the layers or nanoparticles of magnetic metal oxide/oxides (e.g. Fe₃O₄), and so the magnetic properties of the carrier remain unaffected.

This form of regeneration is possible in the case of other types of a carrier according to the invention with cores formed by other transition metals.

In order to achieve the maximum effect of UV radiation, it is advantageous if the carrier is dispersed in an aqueous solution circulating in a closed flow system through a silicone tube, in front of which is situated a source of UV radiation with appropriate wavelength. The flow rate in the system must be selected with respect to the diameter of the silicone tube and the length of the flow system so that the carrier will be exposed to the radiation for at least 25% of the total time.

In another variant, it is also possible to use a batch system, e.g. a suitable vessel with a carrier in an aqueous solution. In that case, it is necessary to use mechanical stirring, e.g., propeller stirring or rotation on a rotator, and avoid using a magnetic stirrer which would cause clustering of the carrier at the point of the action of the magnetic forces.

After the completion of photocatalytic degradation it is then possible to separate the carrier from the aqueous medium by the magnetic field.

For photocatalytic degradation of residues of organic substances it is advantageous to use UV radiation having a wavelength of 200-400 nm, preferably in the range between 260 and 340 nm. In addition, the intensity of UV radiation must be at least 1 mW/cm² of the irradiated area of the carrier, whereby the intensity 60 mW/cm² of the irradiated area of the carrier enables complete decomposition of organic molecules within 80 minutes and the intensity 300 mW/cm² of the irradiated area of the carrier within 12 minutes.

1. A method for separation of biopolymer molecules, particularly biopolymer molecules from the group consisting of mono- and multi-phosphorylated peptides, recombinant peptides/proteins with a polyhistidine tag or another chemically similar biospecific tag, cysteine-containing peptides/proteins and nucleic acids, characterized in that a biopolymer molecule from the group consisting of mono- and multi-phosphorylated peptides, recombinant peptides/proteins with a polyhistidine tag or another chemically similar biospecific tag, cysteine-containing peptides/proteins, nucleic acids, is bound in a binding solution by a specific binding to a carrier which contains a core having dimensions in nano- and/or submicro- and/or microscale, formed by oxide of at least one transition metal, on whose surface is deposited at least one continuous or non-continuous layer and/or nanoparticles of magnetic metal oxide and/or such nanoparticles are deposited in its inner structure, and subsequently undesirable and non-specifically bound components are washed off at least once from the carrier-bound biomolecules by a washing solution, whereupon the biopolymer molecules are eluted from it by changing pH and/or by an elution solution.

2-26. (canceled)
